SEQUENCE LISTING (8 sequences)

SEQ ID NO: 1 - protein, 119 aa, Mus musculus
EIQLQQSGGELVKPGASVKISCTASGYSFTGYNMNWVKQSHGKSPEWIGNINPYYGSTSYNQKFKGKATLTVDKSSSTAYMQLNSLTSEDSAVYYCARWLGLGYAMDYWGQGTSVTVSS

SEQ ID NO: 2 - protein, 112 aa, Mus musculus
DIVLTQSPASLVVSLGQGATISCRASKSVSPSGYSSMHWYQQKPGQPPKLLIYLASNLESGVPARFSGSGSGTDFTLNIHPVEEEDAATYYCQHSRELPWTFGGGTKLEIKR

SEQ ID NO: 3 - protein, 10 aa, Mus musculus
GYSFTGYNMN

SEQ ID NO: 4 - protein, 15 aa, Mus musculus
NINPYYGSTSYNQKF

SEQ ID NO: 5 - protein, 10 aa, Mus musculus
WLGLGYAMDY

SEQ ID NO: 6 - protein, 15 aa, Mus musculus
RASKSVSPSGYSSMH

SEQ ID NO: 7 - protein, 7 aa, Mus musculus
LASNLES

SEQ ID NO: 8 - protein, 9 aa, Mus musculus
QHSRELPWT

(19) United States
(12) Patent Application Publication
(10) Pub. No.: US 2026/0217854 A1
(43) Pub. Date: Jul. 30, 2026

(54) ANTIBODIES SPECIFIC TO TRANSMEMBRANE AND COILED-COIL DOMAIN FAMILY 3 AND USES THEREOF

(71) Applicants: DEVELOPMENT CENTER FOR BIOTECHNOLOGY, New Taipei City (TW); CHANG GUNG MEMORIAL HOSPITAL, LINKOU, TAOYUAN CITY (TW)

(72) Inventors: Cheng-Chou Yu, Taipei City (TW); Shao-Wei Huang, Taipei City (TW); Chuan-Lung Hsu, Taipei City (TW); Alice L. Yu, Taoyuan (TW); John Yu, Taoyuan (TW); Ya-Hui Wang, Taoyuan (TW)

(21) Appl. No.: 19/142,809

(22) PCT Filed: Dec. 29, 2022
(86) PCT No.: PCT/US2022/082524

§ 371 Date: Jun. 24, 2025

(51) Int. Cl. C07K 16/30; A61K 31/40; A61K 38/00; A61K 47/68; A61P 35/00; C12N 5/078; C12N 5/095; C12N 15/63; G01N 33/575; G01N 33/68

(52) U.S. Cl. CPC C07K 16/30; A61K 31/40; A61K 38/00; A61K 47/6851; A61P 35/00; C12N 5/0634; C12N 5/0695; C12N 15/63; G01N 33/575; G01N 33/6893; C07K 2317/31; C07K 2317/33; C07K 2317/565; C12N 2510/00

(57) ABSTRACT

The present disclosure relates to an antibody or antigen-binding fragment thereof that is specific for transmembrane and coiled-coil domain family 3 (TMCC3). The present disclosure also relates to a pharmaceutical composition for treating and/or preventing a disease related to a TMCC3-mediated signal in a subject in need thereof, and a method for detecting TMCC3, a cancer stem cell or a cancer in a sample.

### FIELD OF THE INVENTION

The present disclosure relates to an antibody or antigen-binding fragment thereof, which is specific to transmembrane and coiled-coil domain family 3 (TMCC3), and uses thereof.

### STATEMENT REGARDING SEQUENCE LISTING

The sequence listing associated with this application is provided in text format in lieu of a paper copy and is hereby incorporated by reference into the specification. The name of the XML file containing the sequence listing is sl.xml. The XML file is 8,642 bytes; was created on Jun. 20, 2025; and is being submitted electronically via Patent Center.

### BACKGROUND OF THE INVENTION

Cancer is a leading cause of death and an important barrier to increasing life expectancy worldwide. The majority of cancer deaths occur as a result of recurrent or metastatic disease rather than from the effects of the primary tumor. Accumulating evidences suggest that cancer stem cell (CSC) population, a subgroup of cancer cells, is responsible for the chemoresistance and cancer relapse, as it has ability to self-renew and to differentiate into the heterogeneous lineages of cancer cells.

Since the first identification of CSCs in leukemia in 1994, cancer stem cells have been shown to display the propensity for metastasis and resistance to chemotherapy and radiation, which might contribute to tumor relapse years after the clinical remission (Phi L T H, et al. Stem Cells Int. 2018, 5416923 (2018)). The plasticity of CSCs is reflected also by the large number of signaling pathways that are involved in the induction and maintenance of CSCs (Zhang, S., et al. Identification and characterization of ovarian cancer-initiating cells from primary human tumors. Cancer research 68, 4311-4320 (2008)). Given the functional relationship between CSCs and normal stem cells, the role of signaling pathways involved in the physiology of normal stem cells, such as WNT, Notch, and Hedgehog (Hh), has been investigated with particular attention (Matsui, W. H. Medicine (Baltimore) 95, S8-S19 (2016); Cochrane, C. R. et al. Cancers (Basel) 7, 1554-1585 (2015)). Indeed, many CSC markers are also expressed on normal stem cell populations where they may play essential roles in tissue hemostasis and renewal (Yang, W., et al. Int J Mol Sci 22 (2021); Terraneo, N. et al. Front Oncol 10, 319 (2020)).

Thus, it is important to identify unique molecular markers of CSCs not expressed in normal postnatal stem cells, which facilitate the development of novel CSC-directed therapeutic agents.

### SUMMARY OF THE INVENTION

The present disclosure provides antibodies specific for a novel molecular marker of CSCs not expressed in normal postnatal stem cells and its use for treating or preventing or detecting a cancer.

Accordingly, the present disclosure provides an antibody or antigen-binding fragment thereof that is specific for an epitope in TMCC3; wherein the antibody or antigen-binding fragment thereof comprises complementarity determining regions (CDRs) of a heavy chain variable region and CDRs of a light chain variable region,
wherein the CDRs of the heavy chain variable region comprise:
CDRH1 of an amino acid sequence of SEQ ID NO: 3, CDRH2 of an amino acid sequence of SEQ ID NO: 4, and CDRH3 of an amino acid sequence of SEQ ID NO: 5; and
wherein the CDRs of the light chain variable region comprise:
CDRL1 of an amino acid sequence of SEQ ID NO: 6, CDRL2 of an amino acid sequence of SEQ ID NO: 7, and CDRL3 of an amino acid sequence of SEQ ID NO: 8.

In some embodiments, the heavy chain variable region comprises the amino acid sequence of SEQ ID NO: 1 or a substantially similar sequence having at least 95% sequence identity; and/or the light chain variable region comprises the amino acid sequence of SEQ ID NO: 2 or a substantially similar sequence having at least 95% sequence identity.

In some embodiments of the disclosure, the antibody or antigen-binding fragment thereof is an Fab fragment, an F(ab′)₂ fragment, an ScFv fragment, a monoclonal antibody, a chimeric antibody, a nanobody, a humanized antibody or a human antibody.

In some embodiments of the disclosure, the antibody or antigen-binding fragment thereof is multispecific. In some embodiments of the disclosure, the antibody or antigen-binding fragment thereof is linked to a second antibody or antigen-binding fragment thereof that is specific for a second epitope.

In some embodiments of the disclosure, the antibody or antigen-binding fragment thereof is conjugated with a therapeutic agent. Examples of the therapeutic agent include, but are not limited to antimetabolites, alkylating agents, alkylating-like agents, DNA minor groove alkylating agents, anthracyclines, antibiotics, calicheamicins, antimitotic agents, topoisomerase inhibitors, proteasome inhibitors, and radioisotopes. In some embodiments of the disclosure, the therapeutic agent is selected from DM1, DM3, DM4, monomethyl auristatin E (MMAE), and monomethyl auristatin F (MMAF).

In some embodiments of the disclosure, the antibody or antigen-binding fragment thereof is expressed on a surface of a cell. The cell may be an immune cell, a cancer stem cell or a stem cell. In one embodiment of the disclosure, the immune cell is a T cell.

The present disclosure provides a vector encoding the antibody or antigen-binding fragment thereof as disclosed herein.

The present disclosure provides a genetically engineered cell expressing the antibody or antigen-binding fragment thereof as disclosed herein or containing the vector as disclosed herein.

The present disclosure also provides a method for manufacturing the antibody or antigen-binding fragment thereof as disclosed herein, comprising: (a) introducing into a host cell one or more polynucleotides encoding said antibody or antigen-binding fragment; (b) culturing the host cell under conditions favorable for expression of the one or more polynucleotides; and (c) optionally, isolating the antibody or antigen-binding fragment from the host cell and/or a medium in which the host cell is grown.

The present disclosure provides a pharmaceutical composition comprising the antibody or antigen-binding fragment thereof or the genetically engineered cell as disclosed herein and pharmaceutically acceptable carrier.

The present disclosure provides a vessel or injection device comprising the antibody or antigen-binding fragment thereof or the genetically engineered cell as disclosed herein.

The present disclosure provides a method for treating or preventing a disease related to a TMCC3-mediated signal in a subject in need thereof, comprising administering a therapeutically effective amount of the antibody or antigen-binding fragment thereof or the genetically engineered cell as disclosed herein. Alternatively, the present disclosure provides a pharmaceutical composition for use in treating or preventing a disease related to a TMCC3-mediated signal in a subject in need thereof, comprising a therapeutically effective amount of the antibody or antigen-binding fragment thereof or the genetically engineered cell as used herein and a pharmaceutically acceptable carrier. The present disclosure still also provides a method for treating, prophylactic treating and/or preventing a cancer in a subject afflicted with the cancer, comprising administering to the subject the pharmaceutical composition. In some embodiments of the disclosure, the tumor is a solid tumor. Examples of the tumor include but are not limited to lung cancer, breast cancer, ovary cancer, pancreas cancer, bile duct cancer, gallbladder cancer, prostate cancer, or colorectal cancer. Alternatively, the present disclosure provides a pharmaceutical composition for use in treating, prophylactic treating and/or preventing a cancer in a subject afflicted with the cancer, comprising an effective amount of the antibody or antigen-binding fragment thereof, or the genetically engineered cell as disclosed herein.

In some embodiments of the disclosure, the pharmaceutical composition is in a form suitable for injection. Alternatively, the present disclosure provides a method for administering the antibody or antigen-binding fragment thereof as disclosed herein into the body of a subject comprising injecting the antibody or antigen-binding fragment into the body of the subject.

In some embodiments of the disclosure, the injection is subcutaneous, intravenous or intramuscular. Alternatively, the antibody or antigen-binding fragment is injected into the body of the subject subcutaneously, intravenously, or intramuscularly.

The present disclosure provides a method for detecting TMCC3, a cancer stem cell or a cancer in a sample comprising contacting the sample with the antibody or antigen-binding fragment thereof as disclosed herein.

In some embodiments of the disclosure, the method further comprises assessing a level of expression of TMCC3 in the sample, wherein an increase in the level of expression of TMCC3 as compared to a standard indicates the presence of cancer stem cells in the sample.

Examples of the cancer stem cell include but are not limited to a hematopoietic, epidermal, breast, ovary, lung, pancreas, prostate, brain, colon, bone marrow, or lymph cancer stem cell.

The present disclosure provides a kit for detecting TMCC3 or a cancer in a sample, wherein the kit comprises the antibody or antigen-binding fragment thereof as disclosed herein.

The present disclosure provides a method for detecting a predisposition to a cancer or predicting likelihood, treatment response, prognosis or recurrence of the cancer in a subject, comprising assessing a level of expression of TMCC3 in a sample with the antibody or antigen-binding fragment thereof as disclosed herein, wherein an increase in the level of expression of TMCC3 as compared to a standard indicates the presence of cancer stem cells in the sample.

The present disclosure is described in detail in the following sections. Other characteristics, purposes and advantages of the present disclosure can be found in the detailed description and claims.

### BRIEF DESCRIPTION OF DRAWINGS

FIG. 1 shows the expression level of TMCC3 mRNA in tumor tissue in breast cancer patients. High expression of TMCC3 correlates with poor clinical outcome of breast cancer patients, including early stage patients.

FIG. 2 shows greater TMCC3 expresses in tumor than normal tissues and contributes to cancer progression.

FIG. 3 shows the binding affinity of anti-TMCC3 mAb by ELISA assay.

FIG. 4 shows binding activity of anti-TMCC3 mAb in TMCC3-overexpressing MCF7.

FIG. 5 shows the expression of TMCC3 in breast, pancreatic and ovarian cancer PDX tumors.

FIG. 6A shows FACS analysis with mAb8D6, showing TMCC3 expression on the surface of serous (OC042) and clear cell (OC085) ovarian cancer PDXs. FIG. 6B shows IHC assay of TMCC3 expression was also detected in these PDXs.

FIGS. 7A to 7C show the expression of TMCC3 in primary ovarian cancer specimens. FIG. 7A shows the results of IHC analysis. FIG. 7B shows the expression levels in 3 subtypes of ovarian cancers: mucinous, clear cell and endometrioid type. FIG. 7C shows the expression levels in early stage (stage I-II) and late-stage (stage III-IV).

FIGS. 8A to 8J show survival curves of ovarian cancer patients in relation to TMCC3 expression levels.

FIGS. 9A to 9D show that TMCC3 serves as an enrichment marker for CSCs from breast and ovarian cancer PDXs, assessed by sphere-forming capacity in vitro (A,C), or tumor growth in vivo (B, D).

FIGS. 10A to 10E shows tumor suppression by ADC-8D6 conjugated MMAF in vitro and in vivo.

FIG. 11 shows the result of reduced mass chromatography analysis of Tri-mannosyl-anti-TMCC3 8D6 mAb.

FIGS. 12A to 12B shows the result of reduced mass chromatography analysis of anti-TMCC3 8D6 mAb-4Az.

FIG. 13 shows the result of reduced mass chromatography analysis of anti-TMCC3 8D6 mAb-4(DBCO-vc-MMAE).

FIG. 14 shows clinical information of ovarian cancer patient OC085 and TMCC3 expression of her tumors.

FIGS. 15A to 15G show anti-cancer activity of ADC-tri-mannosyl-MMAE (8D6) against OC085 PDX in vivo.

FIGS. 16A to 16B show anti-cancer activity of tri-mannosyl ADC (8 mpk) against OC085 PDX in vivo.

FIGS. 17A to 17B show anti-cancer activity of chemotherapy and ADC-8D6 (8 mg/kg/dose) against OC085 PDX in vivo.

FIGS. 18A to 18B show that 50 mpk of niraparib failed to suppress the tumor growth of OC085 PDX in vivo.

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise.

The term “antibody”, as used herein, refers to any antigen-binding molecule or molecular complex comprising at least one complementarity determining region (CDR) that is specific for or interacts with a particular antigen (TMCC3). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-SARS-CoV-2-Spike protein antibody (or antigen-binding portion thereof) may be identical to the human germline sequences, or may be naturally or artificially modified. An amino acid consensus sequence may be defined based on a side-by-side analysis of two or more CDRs.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

As used herein, the term “being specific to” or “binding specifically to” means that an antibody does not cross react to a significant extent with other epitopes.

As used herein, the term “epitope” refers to the site on the antigen to which an antibody binds.

As used herein, the term “complementarity determining region” (CDR) refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

As applied to polypeptides, the term “substantial similarity” or “substantially similar” means that two peptide sequences, when optimally aligned, such as by the programs GAP or BESTFIT using default gap weights, share at least 90%, 91%, 92%, 93%, 94%, 95%, 96%, 97%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

The term “monoclonal antibody” as used herein is not limited to antibodies produced through hybridoma technology. A monoclonal antibody is derived from a single clone, including any eukaryotic, prokaryotic, or phage clone, by any refers to available or known in the art.

The term “chimeric” antibody as used herein refers to an antibody having variable sequences derived from a non-human immunoglobulin and human immunoglobulin constant regions, typically chosen from a human immunoglobulin template.

As used herein, the term “nanobody” refers to an antibody comprising the small single variable domain (VHH of antibodies obtained from camelids and dromedaries. Antibody proteins obtained from members of the camel and dromedary (Camelus bactrianus and Camelus dromaderius) family including new world members such as llama species (Lama pacos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

“Humanized” forms of non-human antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence.

As used herein, the term “nanobody” refers to an antibody comprising the small single variable domain (VHH of antibodies obtained from camelids and dromedaries. Antibody proteins obtained from members of the camel and dromedary (Camelus bactrianus and Camelus dromaderius) family including new world members such as llama species (Lama pacos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

As used in the present disclosure, the term “therapeutic agent” refers to any compound, substance, drug, drug or active ingredient having a therapeutic or pharmacological effect that is suitable for administration to a mammal, for example a human.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “T cell” includes CD4⁺ T cells and CD8⁺ T cells. The term T cell also includes T helper 1 type T cells, T helper 2 type T cells, T helper 17 type T cells and inhibitory T cells.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

As used herein, the term “immunoconjugate” refers to an antigen-binding protein, e.g., an antibody or antigen-binding fragment, which is chemically or biologically linked to a radioactive agent, a cytokine, an interferon, a target or reporter moiety, an enzyme, a peptide or protein or a therapeutic agent. The antigen-binding protein may be linked to the radioactive agent, cytokine, interferon, target or reporter moiety, enzyme, peptide or therapeutic agent at any location along the molecule so long as it is able to bind its target (TMCC3). Examples of immunoconjugates include antibody-drug conjugates and antibody-toxin fusion proteins. In one embodiment of the invention, the agent may be a second, different antibody that binds specifically to TMCC3. The type of therapeutic moiety that may be conjugated to the anti-TMCC3 protein (e.g., antibody or fragment) will take into account the condition to be treated and the desired therapeutic effect to be achieved.

As used herein, the term “vector” is intended to refer to a nucleic acid molecule capable of transporting another nucleic acid to which it has been linked.

The term “genetically engineered” or “genetic engineering” of cells refers to manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

As used in the present invention, the term “pharmaceutical composition” refers to a mixture containing a therapeutic agent administered to a mammal, for example a human, for preventing, treating, or eliminating a particular disease or pathological condition that the mammal suffers.

As used herein, the term “therapeutically effective amount” or “efficacious amount” refers to the amount of an antibody that, when administered to a mammal or other subject for treating a disease, is sufficient to affect such treatment for the disease.

As used herein, the terms “treatment,” “treating,” and the like, cover any treatment of a disease in a mammal, particularly in a human, and include: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc. Particularly, the subject is vaccinated.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

“Cancer,” “tumor,” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

As used herein the term “cancer stem cell” refers to a cell that is capable of self-renewal and differentiating into the lineages of cancer cells that comprise a solid tumor and/or hematological malignancy. Cancer stem cells are uniquely able to initiate and sustain the disease. Variant neoplastic stem cells are pluripotent cells which exhibit the properties of cancer stem cells.

As used herein, the term “sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

The present disclosure develops an antibody or antigen-binding fragment thereof that is specific for an epitope in TMCC3.

Particularly, the antibody or antigen-binding fragment thereof that is specific for an epitope in TMCC3; wherein the antibody or antigen-binding fragment thereof comprises CDRs of a heavy chain variable region and CDRs of a light chain variable region, wherein the CDRs of the heavy chain variable region comprise CDRH1, CDRH2, and CDRH3 regions, and the CDRs of the light chain variable region comprise CDRL1, CDRL2, and CDRL3 regions, and wherein:
the CDRH1 region comprises the amino acid sequence of SEQ ID NO: 3 or a substantially similar sequence having at least 90%, at least 95%, at least 98% or at least 99% sequence identity; the CDRH2 region comprises the amino acid sequence of SEQ ID NO: 4 or a substantially similar sequence having at least 90%, at least 95%, at least 98% or at least 99% sequence identity; the CDRH3 region comprises the amino acid sequence of SEQ ID NO: 5 or a substantially similar sequence having at least 90%, at least 95%, at least 98% or at least 99% sequence identity; and the CDRL1 region comprises the amino acid sequence of SEQ ID NO: 6 or a substantially similar sequence having at least 90%, at least 95%, at least 98% or at least 99% sequence identity; the CDRL2 region comprises the amino acid sequence of SEQ ID NO: 7 or a substantially similar sequence having at least 90%, at least 95%, at least 98% or at least 99% sequence identity; the CDRL3 region comprises the amino acid sequence of SEQ ID NO: 8 or a substantially similar sequence having at least 90%, at least 95%, at least 98% or at least 99% sequence identity.

In some embodiments, the antibody or antigen-binding fragment thereof comprises a heavy chain variable region comprising the amino acid sequence of SEQ ID NO: 1 or a substantially similar sequence thereof; and/or a light chain variable region comprising the amino acid sequence of SEQ ID NO: 2 or a substantially similar sequence thereof. In some further embodiments, the antibody or antigen-binding fragment thereof comprises a heavy chain variable region comprising the amino acid sequence of SEQ ID NO: 1 or a substantially similar sequence thereof having at least 90%, at least 95%, at least 98% or at least 99% sequence identity. In some further embodiments, the antibody or antigen-binding fragment thereof comprises a light chain variable region comprising the amino acid sequence of SEQ ID NO: 2 or a substantially similar sequence thereof having at least 90%, at least 95%, at least 98% or at least 99% sequence identity.

The sequences in the disclosure are shown in Table 1.

TABLE 1

| | | SEQ |
|---|---|---|
| | Sequence | ID NO. |
| TMCC3 | EIQLQQSGGELVKPGASVKISCTASGYSFTGYNM | 1 |
| 8D6 VH | NWVKQSHGKSPEWIGNINPYYGSTSYNQKFKGKA | |
| | TLTVDKSSSTAYMQLNSLTSEDSAVYYCARWLGL | |
| | GYAMDYWGQGTSVTVSS | |
| TMCC3 | DIVLTQSPASLVVSLGQGATISCRASKSVSPSGY | 2 |
| 8D6 VL | SSMHWYQQKPGQPPKLLIYLASNLESGVPARFSG | |
| | SGSGTDFTLNIHPVEEEDAATYYCQHSRELPWTF | |
| | GGGTKLEIKR | |
| HCDR 1 | GYSFTGYNMN | 3 |
| HCDR 2 | NINPYYGSTSYNQKF | 4 |
| HCDR 3 | WLGLGYAMDY | 5 |
| LCDR 1 | RASKSVSPSGYSSMH | 6 |
| LCDR 2 | LASNLES | 7 |
| LCDR 3 | QHSRELPWT | 8 |

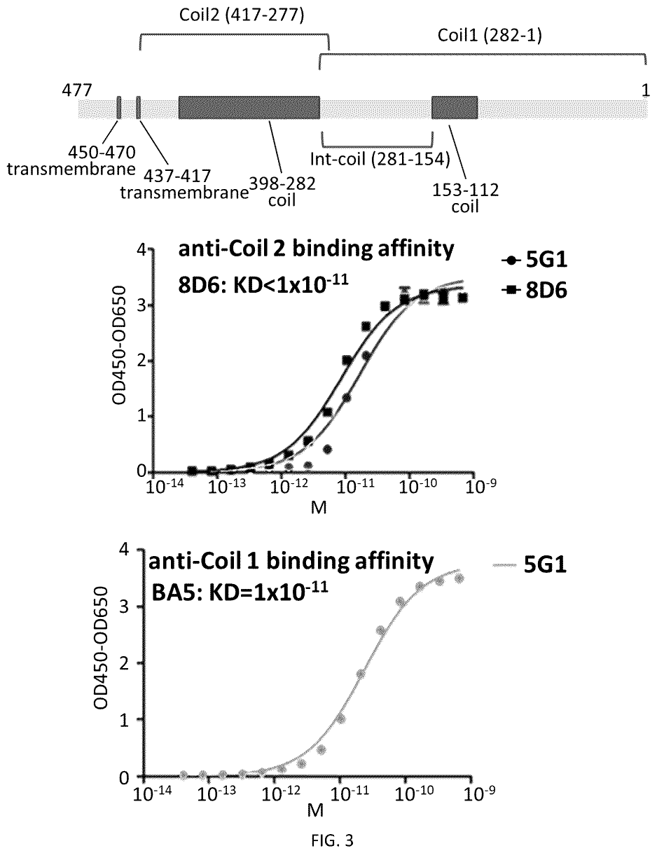

In some embodiments of the disclosure, greater phosphorylated TMCC3 at Serine 216 is noted in bCSCs than in non-bCSCs. TMCC3 belongs to the TMCC family that includes TMCC1-3 and contains two coiled-coil domains of the N-terminal region and two transmembrane domains of the C-terminal region. In some embodiments of the disclosure, TMCC3 expression is strongest in lung metastatic lesions, followed by metastatic lymph nodes, and then primary tumor of breast cancer. Silencing of TMCC3 decreases CSCs as reflected by reduced mammosphere formation and ALDH activities in vitro, along with reduced tumor growth in vivo. In some embodiments of the disclosure, higher mRNA level of TMCC3 is observed in metastatic lymph nodes than primary tumor of ovarian cancer. TMCC3 knockdown reduces sphere forming capacity in vitro (FIG. 3B) and suppressed tumor growth of ovarian cancer in vivo. TMCC3 overexpression increased ALDH activity in ovarian cancer cells.

The antibody according to the disclosure can be full-length (for example, an IgG1 or IgG4 antibody) or may comprise only an antigen-binding portion (for example, a Fab, F(ab′)₂ or scFv fragment), and may be modified to affect functionality as needed.

The antibody also includes an antigen-binding fragment of a full antibody molecule. An antigen-binding fragment of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of an antigen-binding fragment include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed by the expression “antigen-binding fragment,” as used herein.

An antigen-binding fragment of an antibody typically comprises at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (v) V_{H}-C_{H1}-C_{H2}-C_{H3}, (vi) V_{H}-C_{H2}-C_{H3}; (vii) V_{H}-CL; (viii) V_{L}-C_{H1}; (ix) V_{L}-C_{H2}; (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed here, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The anti-TMCC3 antibody disclosed herein may comprise one or more amino acid substitutions, insertions, and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes an antibody, and an antigen-binding fragment thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another mammalian germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, could easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired properties such as, improved binding specificity, increased binding affinity, improved or enhanced antagonistic or agonistic biological properties (as the case may be), reduced immunogenicity, etc. Antibodies and antigen-binding fragments obtained in this general manner are encompassed by the present disclosure.

The present disclosure also includes an anti-TMCC3 antibody comprising variants of any of the V_{H}, V_{L}, and/or CDR amino acid sequences disclosed herein having one or more conservative substitutions. For example, the present disclosure includes an anti-TMCC3 antibody having V_{H}, V_{L}, and/or CDR amino acid sequences with, e.g., 10 or fewer, 8 or fewer, 6 or fewer, 4 or fewer, etc. conservative amino acid substitutions relative to any of the V_{H}, V_{L}, and/or CDR amino acid sequences disclosed herein.

In some embodiments of the disclosure, the antibody according to the disclosure is a humanized antibody. In order to improve the binding affinity of the humanized antibody according to the disclosure, some amino acid residues in the human framework region are replaced by the corresponding amino acid residues in the species of CDRs; e.g. a rodent.

The antibodies of the present disclosure may be monospecific, bi-specific, or multispecific. Multispecific antibodies may be specific for different epitopes of one target polypeptide or may contain antigen-binding domains specific for more than one target polypeptide. In some embodiments of the disclosure, the antibody or antigen-binding fragment thereof is multispecific. In some embodiments of the disclosure, the antibody or antigen-binding fragment thereof is linked to a second antibody or antigen-binding fragment thereof that is specific for a second epitope. The anti-TMCC3 antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present disclosure includes bi-specific antibodies wherein one arm of an immunoglobulin is specific for TMCC3 or a fragment thereof, and the other arm of the immunoglobulin is specific for a second therapeutic target or is conjugated to a therapeutic moiety.

In some embodiments of the disclosure, the antibody or antigen-binding fragment thereof is conjugated with a therapeutic agent. Examples of the therapeutic agent include, but are not limited to antimetabolites, alkylating agents, alkylating-like agents, DNA minor groove alkylating agents, anthracyclines, antibiotics, calicheamicins, antimitotic agents, topoisomerase inhibitors, proteasome inhibitors, and radioisotopes. In some embodiments of the disclosure, the therapeutic agent is selected from DM1, DM3, DM4, monomethyl auristatin E (MMAE), and monomethyl auristatin F (MMAF).

In one embodiment of the disclosure, the antibody or antigen-binding fragment thereof is expressed on the surface of a cell. Particularly, the cell is an immune cell such as a T-cell, a cancer stem cell or a stem cell.

In some embodiments of the disclosure, the antibody or antigen-binding fragment thereof is in a form of chimeric antigen receptor.

The term “chimeric antigen receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. The generation and construction of CAR are generalized by Jayaraman et al., EBioMedicine 58 (2020) 102931; Zhang et al., Biomarker Research (2017) 5:22; Feins et al., Am J Hematol. (2019) 94: S3-S9; and Roselli et al., J Clin Invest. 2021; 131(2):e142030.

In another aspect, the present disclosure provides a genetically engineered cell expressing the antibody or antigen-binding fragment thereof or containing the vector. The genetically engineered cell may be an immune cell.

In one preferred embodiments of the disclosure, the antibody or antigen-binding fragment thereof can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)).

An example of a method for manufacturing the antibody or antigen-binding fragment comprises: (a) introducing into a host cell one or more polynucleotides encoding said antibody or antigen-binding fragment; (b) culturing the host cell under conditions favorable to expression of the one or more polynucleotides; and (c) optionally, isolating the antibody or antigen-binding fragment from the host cell and/or a medium in which the host cell is grown.

A vector can be used to introduce a polynucleotide encoding the antibody or antigen-binding fragment of the invention to a host cell. In one embodiment, one type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The disclosure provides pharmaceutical compositions comprising the antibody or antigen-binding fragment thereof or the genetically engineered cell. The pharmaceutical compositions of the disclosure are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. When an antibody of the present disclosure is used for treating a condition or disease associated with EPHA10 in an adult patient, it may be advantageous to intravenously administer the antibody of the present disclosure. Depending on the severity of the condition, the frequency and the duration of the treatment can be adjusted. Effective dosages and schedules for administering the antibody may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a vessel or injection device such as standard needle and syringe. In addition, with respect to subcutaneous delivery, a pen delivery device readily has applications in delivering the pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described here in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The present disclosure provides a method for treating or preventing a disease related to a TMCC3-mediated signal in a subject in need thereof, comprising administering a therapeutically effective amount of the antibody or antigen-binding fragment thereof or the genetically engineered cell as disclosed herein. Alternatively, the present disclosure provides a pharmaceutical composition for use in treating or preventing a disease related to a TMCC3-mediated signal in a subject in need thereof, comprising a therapeutically effective amount of the antibody or antigen-binding fragment thereof or the genetically engineered cell as used herein and a pharmaceutically acceptable carrier.

The present disclosure still also provides a method for treating, prophylactic treating and/or preventing a cancer in a subject afflicted with the cancer, comprising administering to the subject the pharmaceutical composition. In some embodiments of the disclosure, the tumor is a solid tumor. Examples of the tumor include but are not limited to lung cancer, breast cancer, ovary cancer, pancreas cancer, bile duct cancer, gallbladder cancer, prostate cancer, or colorectal cancer. Alternatively, the present disclosure provides a pharmaceutical composition for use in treating, prophylactic treating and/or preventing a cancer in a subject afflicted with the cancer, comprising an effective amount of the antibody or antigen-binding fragment thereof, or the genetically engineered cell as disclosed herein.

The present disclosure provides a method for detecting TMCC3, a cancer stem cell or a cancer in a sample comprising contacting the sample with the antibody or antigen-binding fragment thereof as disclosed herein.

In some embodiments of the disclosure, the method further comprises assessing a level of expression of TMCC3 in the sample, wherein an increase in the level of expression of TMCC3 as compared to a standard indicates the presence of cancer stem cells in the sample.

Examples of the cancer stem cell include but are not limited to a hematopoietic, epidermal, breast, ovary, lung, pancreas, prostate, brain, colon, bone marrow, or lymph cancer stem cell.

The present disclosure provides a kit for detecting TMCC3 or a cancer in a sample, wherein the kit comprises the antibody or antigen-binding fragment thereof as disclosed herein.

The anti-TMCC3 antibody of the present disclosure may also be used to detect and/or measure a cancer, or TMCC3-expressing cells in a sample, e.g., for diagnostic purposes. For example, an anti-TMCC3 antibody, or fragment thereof, may be used to diagnose a condition or disease characterized by coronavirus infection. Exemplary diagnostic assays for coronavirus may comprise, e.g., contacting a sample, obtained from a patient, with an antiTMCC3 antibody of the disclosure, wherein the anti-TMCC3 antibody is labeled with a detectable label or reporter molecule. Alternatively, an unlabeled anti-TMCC3 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure coronavirus in a sample include enzyme-linked immunosorbent assay (ELISA), radioimmunoassay (RIA), and fluorescence-activated cell sorting (FACS).

The present disclosure provides a method for detecting a predisposition to a cancer or predicting likelihood, treatment response, prognosis or recurrence of the cancer in a subject, comprising assessing a level of expression of TMCC3 in a sample with the antibody or antigen-binding fragment thereof as disclosed herein, wherein an increase in the level of expression of TMCC3 as compared to a standard indicates the presence of cancer stem cells in the sample.

The following examples are provided to aid those skilled in the art in practicing the present disclosure.

### Example 1 Clinical Relevance of Expression of TMCC3 mRNA in Cancer Tissues

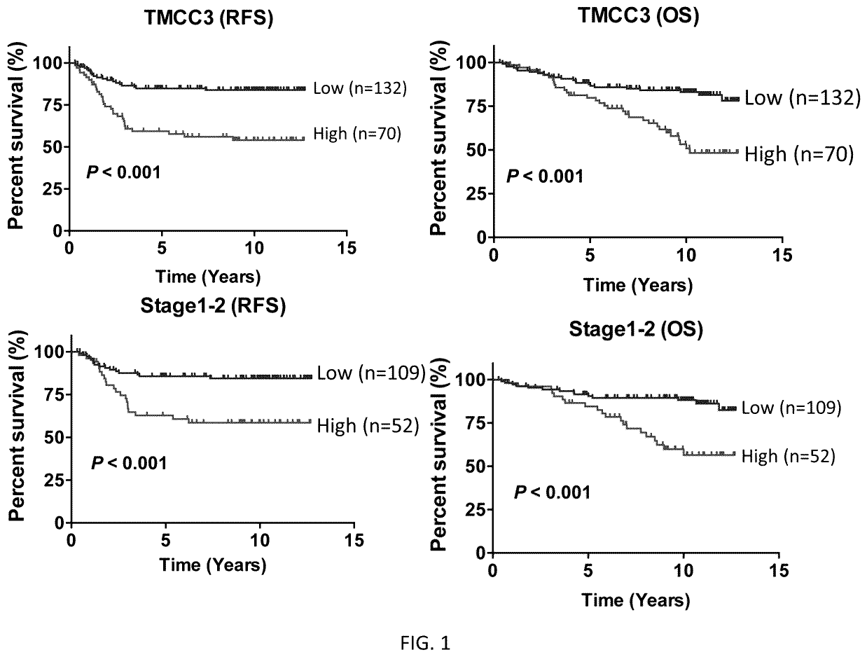

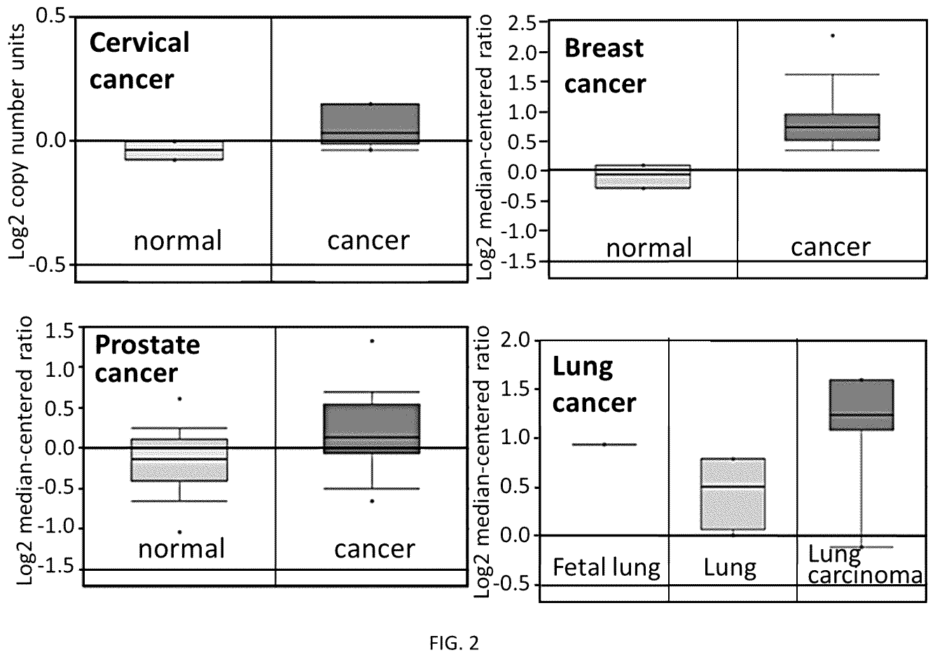

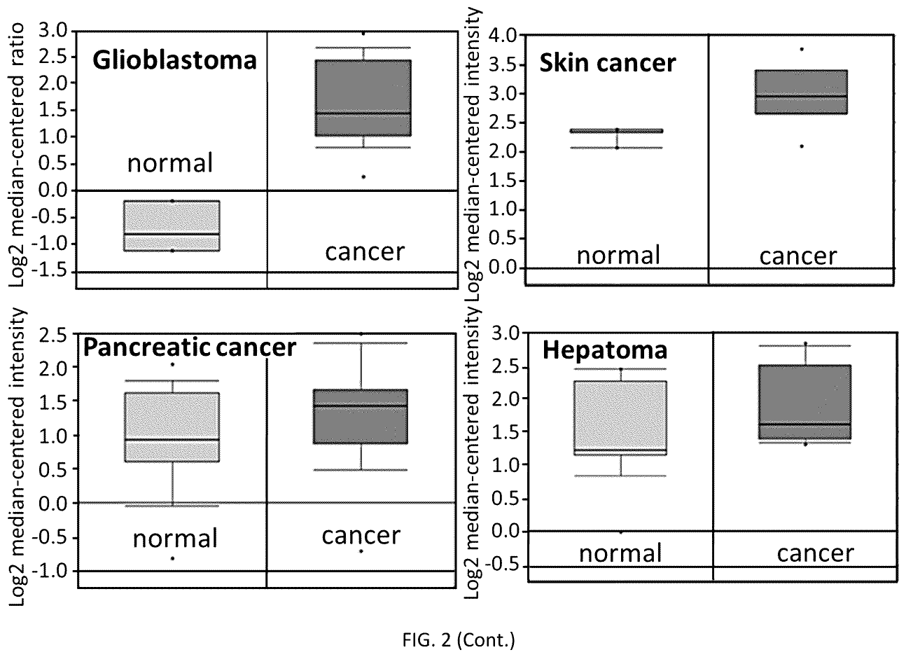

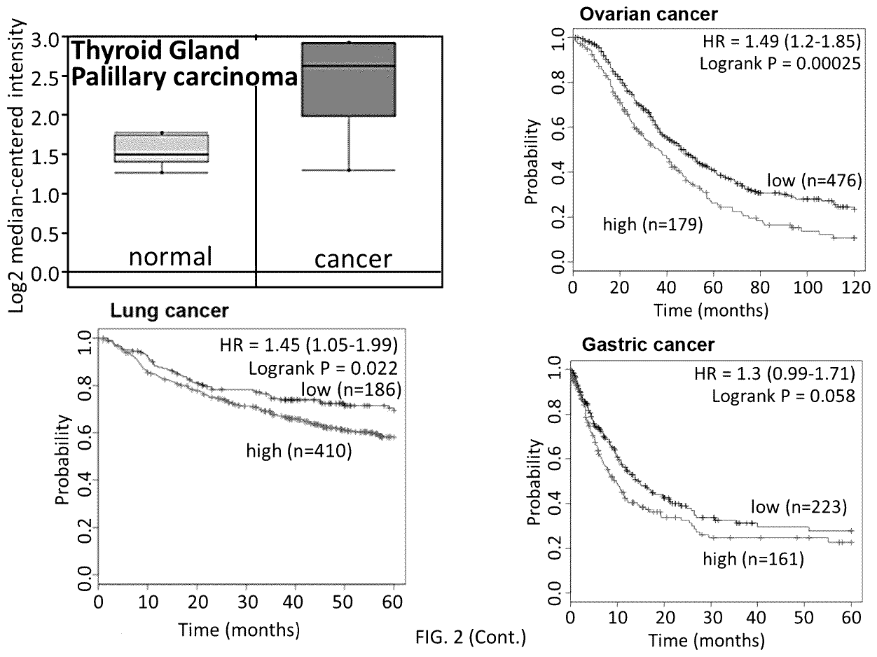

To evaluate the clinical relevance of TMCC3 expression, we examined the mRNA levels of TMCC3 in tumor specimens of 202 patients with breast cancer by qRT-PCR. Kaplan-Meier analyses and a log-rank test showed that patients with low expression level of TMCC3 in tumor had significantly greater relapse free survival (RFS) and overall survival (OS) than patients with high expression levels. This was also true for the 161 patients with early stage breast cancer. Multivariate analyses indicate that the expression level of TMCC3 in tumor tissue is an important and independent predictor for RFS and OS in breast cancer patients (FIG. 1). Using an online RNA microarray database at the ONCOMINE website, we found higher mRNA level of TMCC3 in cancer part than normal tissue in cervical cancer, prostate cancer, pancreatic cancer, lung cancer, glioblastoma, skin cancer, hepatoma and thyroid gland papillary carcinoma. Using Kaplan-Meier Plotter website to evaluate the clinical significance of TMCC3 in cancers, we showed that higher expression of TMCC3 correlated with poor overall survival of patients with ovarian, lung and gastric cancer (FIG. 2). These findings lend further support for the crucial roles of TMCC3 in ovarian cancer progression, and for developing strategies to target TMCC3 for cancer therapy.

### Example 2 Generation of Anti-TMCC3 Monoclonal Antibody

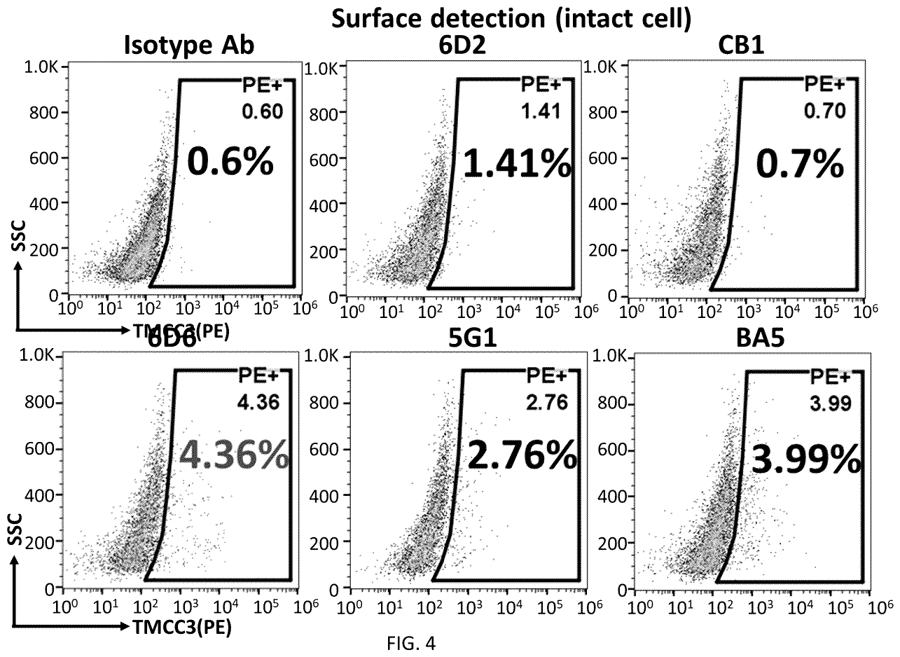

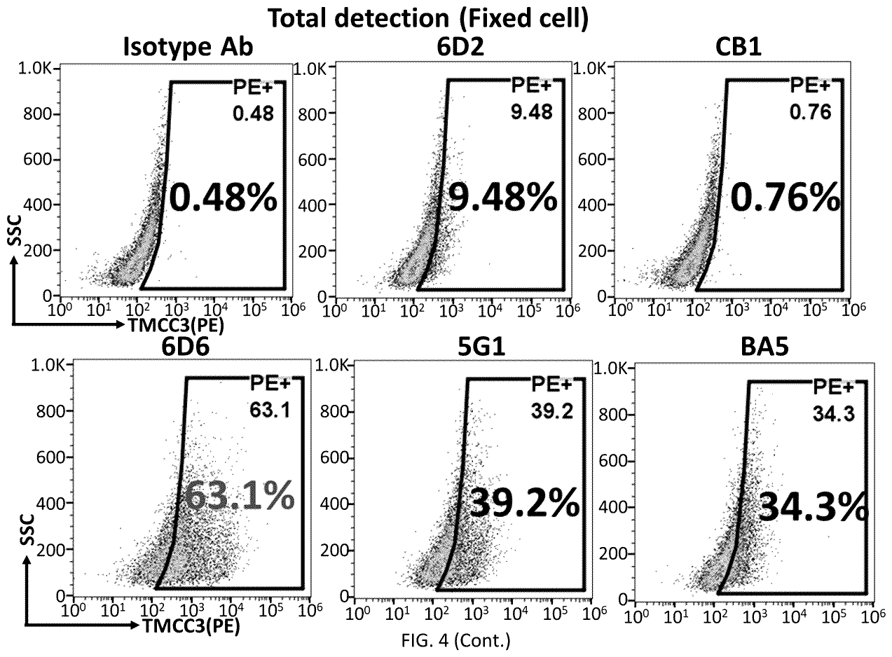

Our endeavors have supported the notion that TMCC3 is a potential theranostic target for clinical prognostication and eradication of CSCs. To generate suitable mAbs for further in depth investigation of this novel protein, mice were immunized with extracellular domain of TMCC3. The binding activity and specificity of anti-TMCC3 mAbs were verified with ELISA and FACS analysis. Coil 1 and coil 2 domain of TMCC3 proteins were generated for ELISA assay. We found clone BA5 recognized the coil 1 domain and clone 8D6 and 5G1 recognized the coil 2 domain of TMCC3 protein, with K_{D} value of 1×10⁻¹¹ M and <1×10⁻¹¹ M, respectively (FIG. 3). Furthermore, fresh and formalin-fixed MCF7 cells overexpressing TMCC3 were prepared for FACS assay to verify the binding activity of mAbs for cell surface or intracellular TMCC3. Among many mAb clones generated, clone 8D6 has the highest binding affinity for both surface and intracellular TMCC3 in MCF7 cells overexpressing TMCC3 (4.36% and 63.1%, respectively), as compared with other clones (FIG. 4).

### Example 3 Sequence of Mouse Anti-TMCC3 8D6 Monoclonal Antibody

A method for obtaining a mouse anti-TMCC3 antibody is as follows. First, a hybridoma of anti-TMCC3 was generated. Such a hybridoma may be generated with standard protocols for the production of monoclonal antibodies. The total RNA of the hybridoma was then isolated, for example using the TRIZOL® reagent. Then, cDNA was synthesized from the total RNA, for example using a first strand cDNA synthesis kit (Superscript III) and an oligo (dT₂₀) primer or an Ig-3′ constant region primer.

Heavy and light chain variable regions of the immunoglobulin genes were then cloned from the cDNA. For example, the VH and VL variable regions of the anti-TMCC3 mAb were amplified from mouse TMCC3 hybridoma cDNAs by PCR, using a mouse Ig-5′ primer set. The PCR products may be cloned directly into a suitable vector (e.g., a pJET1.2 vector) using CLONEJET™ PCR Cloning Kit. The pJET1.2 vector contains lethal insertions and will survive the selection conditions only when the desired gene is cloned into this lethal region. This facilitates the selection of recombinant colonies. Finally, the recombinant colonies were screened for the desired clones, the DNAs of those clones were isolated and sequenced. The immunoglobulin (IG) nucleotide sequences may be analyzed at the international ImMunoGeneTics information system (IGMT) website.

### Example 4 FACS and IHC Analysis of TMCC3 Protein Expression in PDX Tumors

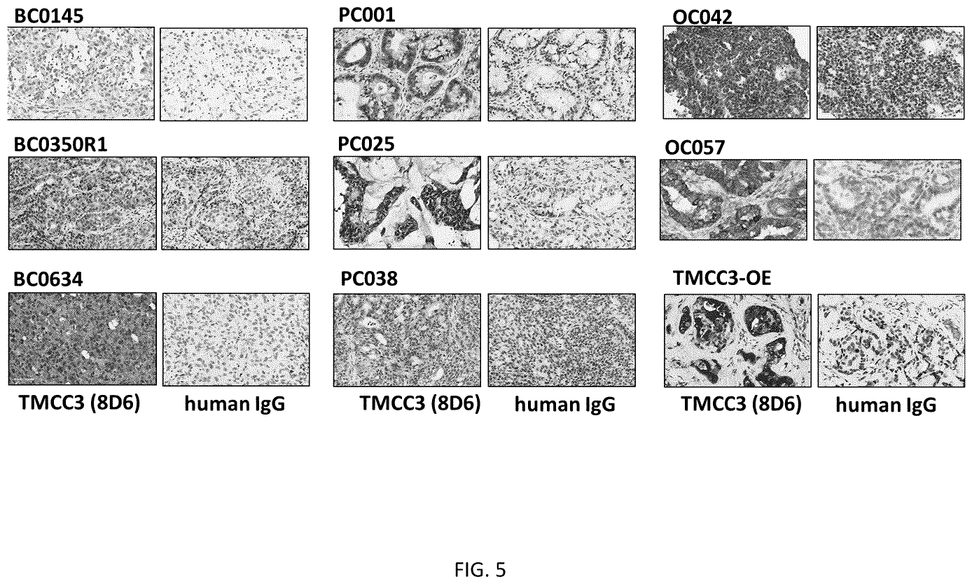

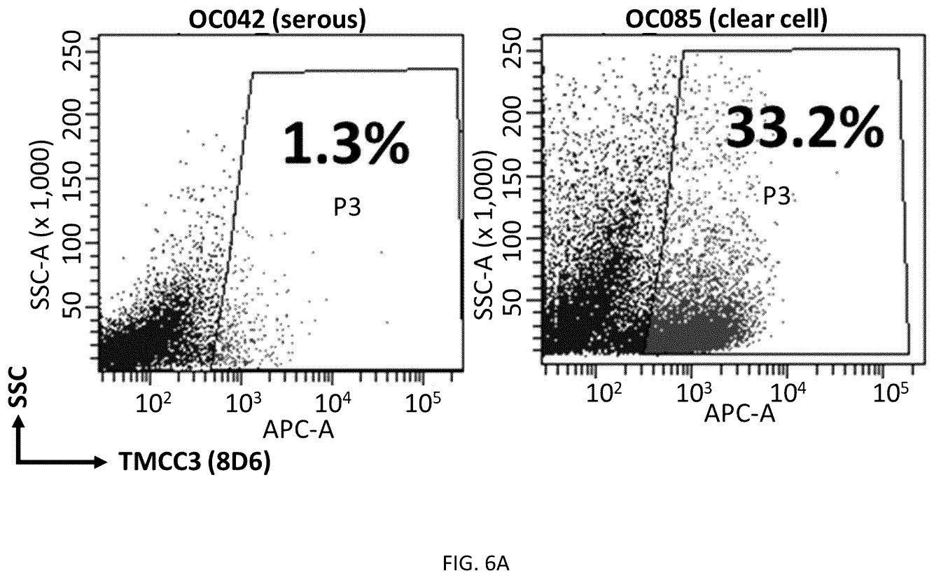

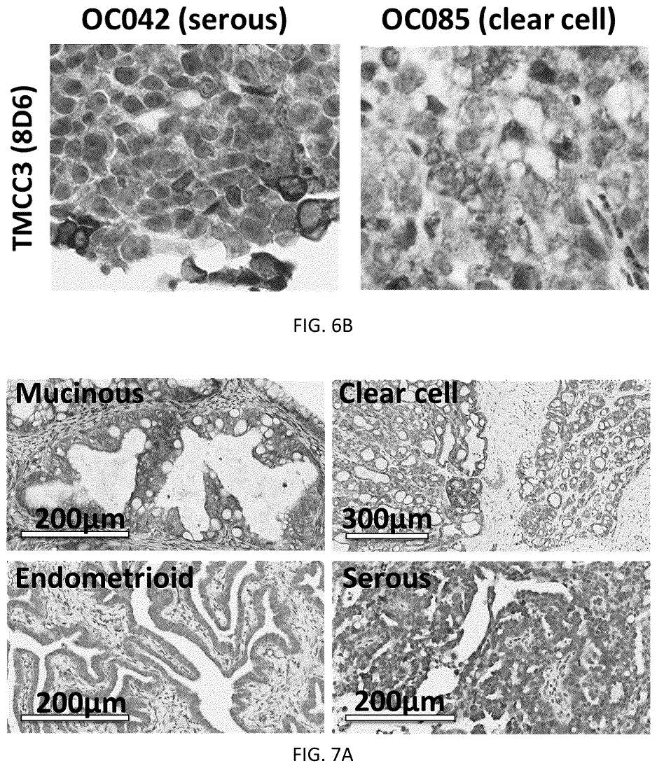

TMCC3 protein expression in breast cancer (BC0145, BC0350R1 and BC0634), pancreatic cancer (PC001, PC025 and PC038) and ovarian cancer (OC042 and OC057) PDXs was detectable using mAb8D6 (FIG. 5). FACS analysis with mAb8D6 showed TMCC3 expression on the surface of serous (OC042) and clear cell (OC085) ovarian cancer PDXs (FIG. 6A). TMCC3 expression was also detected in these PDXs by IHC assay (FIG. 6B).

### Example 5 the Expression of TMCC3 in Primary Ovarian Cancer Specimens

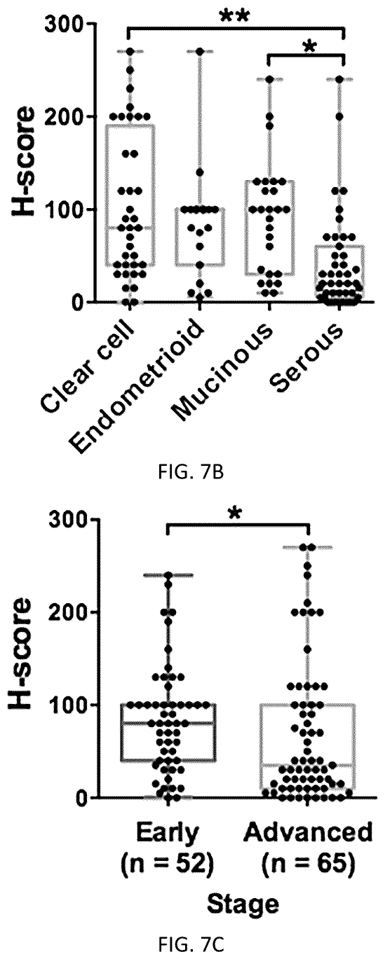

To address the clinical relevance of TMCC3 in ovarian cancer, we examined TMCC3 protein expression in 125 primary ovarian cancer specimens by IHC analysis and its correlation with clinical features and patient outcome. Our results showed that TMCC3 was highly expressed in tumor epithelial cells of mucinous, clear cell and endometrioid type of ovarian cancer tissues (FIG. 7A). Serous type of ovarian cancer tissues expressed lower TMCC3 protein, as compared with the other subtypes of ovarian cancer (FIG. 7B). In TMCC3-positive specimens, the TMCC3 was detected predominantly in the cytoplasm and membrane of tumor cells (FIG. 7A). In particular, clear cell carcinomas showed higher TMCC3 expression than the other subtypes (FIG. 7A). As shown in FIG. 7C, higher expression of TMCC3 was found in early stage (stage I-II) than late-stage (stage III-IV) ovarian cancer tissues (P<0.05).

### Example 6 High Expression Levels of TMCC3 Correlate with Poor Clinical Outcome in Ovarian Cancer

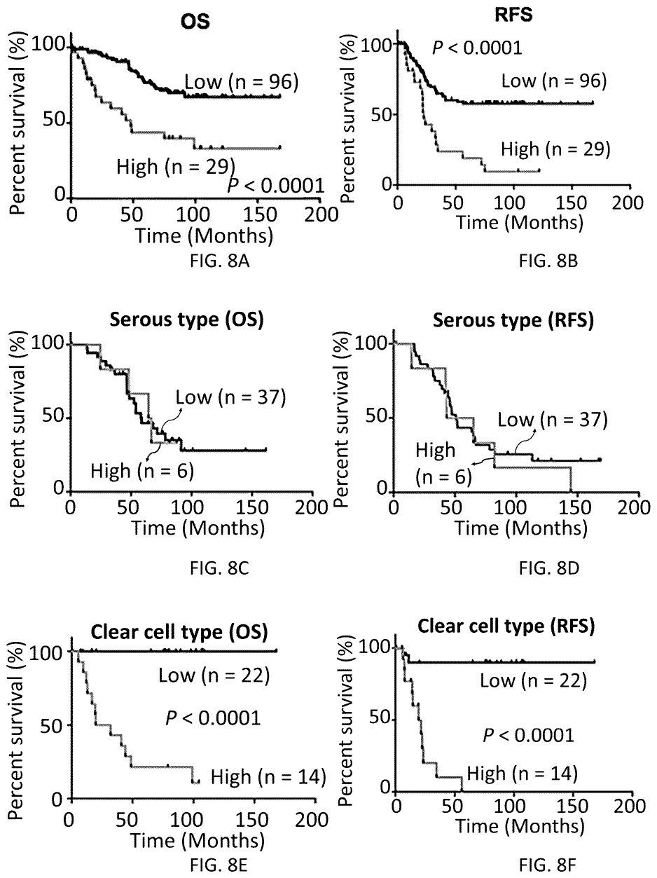

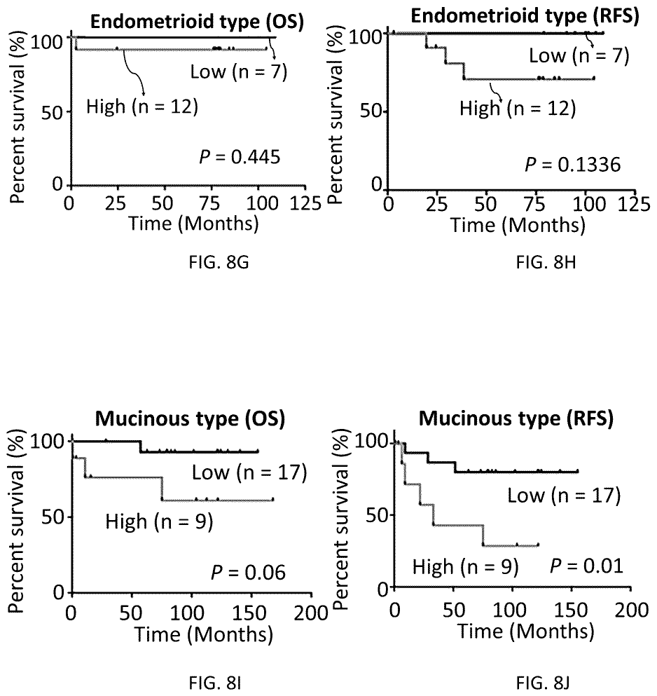

We investigated whether high expression of TMCC3 was a significant predictor of prognosis of ovarian cancer. Our results showed that patients with low expression level of TMCC3 in tumor had significantly greater OS (P<0.0001) and RFS (P<0.0001) than patients with high expression levels (FIGS. 8A and 8B). We then analyzed the potential prognostic value of TMCC3 expression in patients with different subtypes. As shown in FIGS. 8H and 8J, patients with endometrium or mucinous subtype, whose tumors expressed low level of TMCC3 had greater RFS than patients with high level (P=0.1336 & P=0.01, respectively). The survival benefit of patients with low expression of TMCC3 was even more striking for patients with clear cell carcinoma (OS & RFS, P<0.0001 & P<0.0001, respectively) (FIGS. 8E and 8F). These results demonstrated the adverse impacts of high expression of TMCC3 on clinical outcome of ovarian cancer, especially for those with clear cell carcinoma.

### Example 7 CSC Enrichment of Breast and Ovarian Cancer PDXs by Anti-TMCC3 mAbs

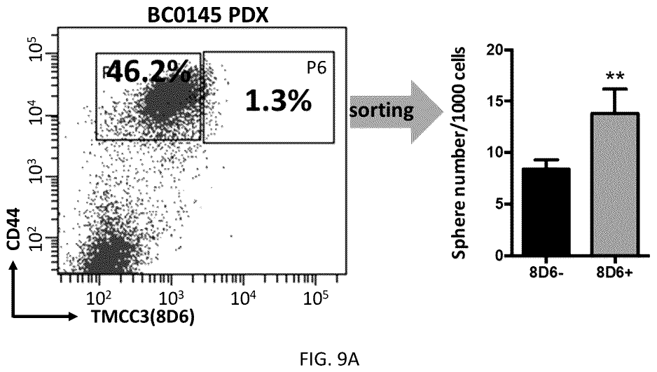

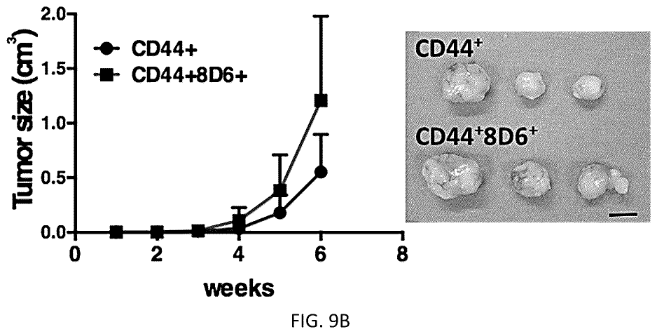

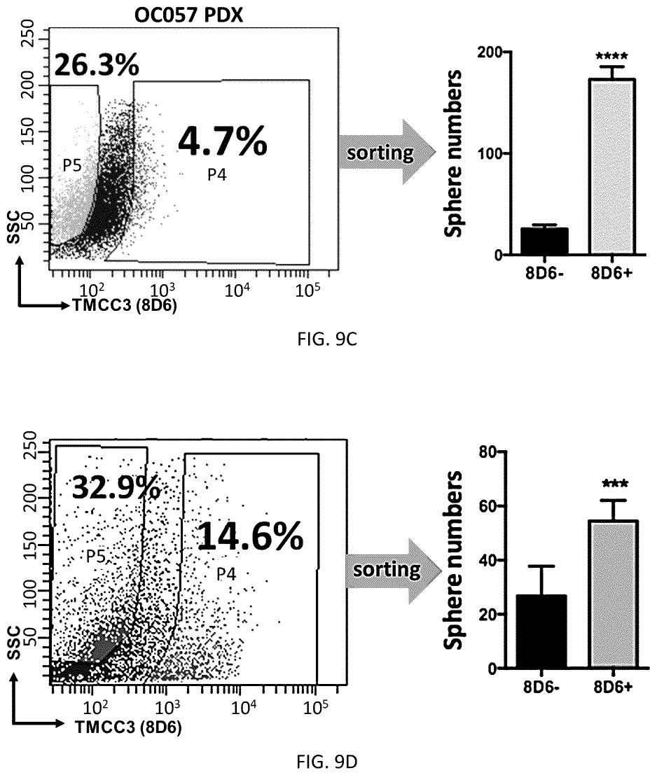

As shown in FIG. 9A, mAb8D6 could bind to BCSC population (CD44⁺, H2k^{d-}, 7AAD⁻) in BC0145 PDX tumor and enrich BCSC population from BC0145 PDX tumors, as reflected by greater in vitro mammosphere forming capacity of 8D6⁺ BCSCs than 8D6⁻ BCSCs. In addition, CD44⁺/TMCC3⁺/H2k^{d-}/7AAD⁻ cells yielded tumors that grew faster and bigger, than CD44⁺/TMCC3⁻/H2k^{d-}/7AAD⁻ cells (FIG. 9B). Similarly, mAb8D6 could enrich CSC population from ovarian cancer PDX tumors, OC057 and OC085. As shown in FIGS. 9C and 9D, 8D6⁺ cells have greater sphere forming capacity than 8D6 cells of OC057 & OC085 PDXs. These findings indicate that mAb8D6 can enrich breast and ovarian CSCs from PDX tumors.

### Example 8 Anti-Cancer Efficacy of a Prototype Anti-TMCC3 Antibody-Drug Conjugate (ADC)

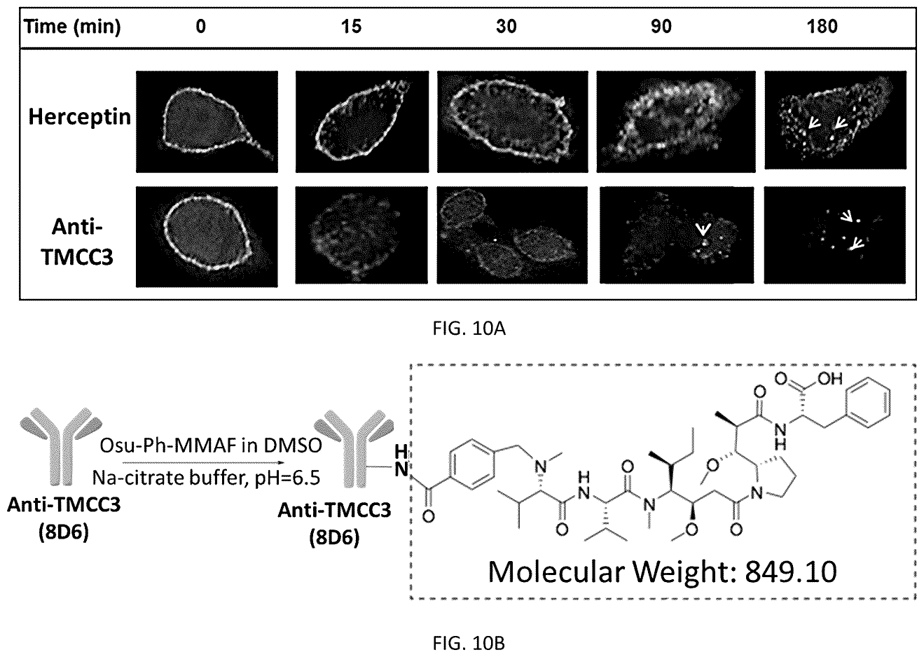

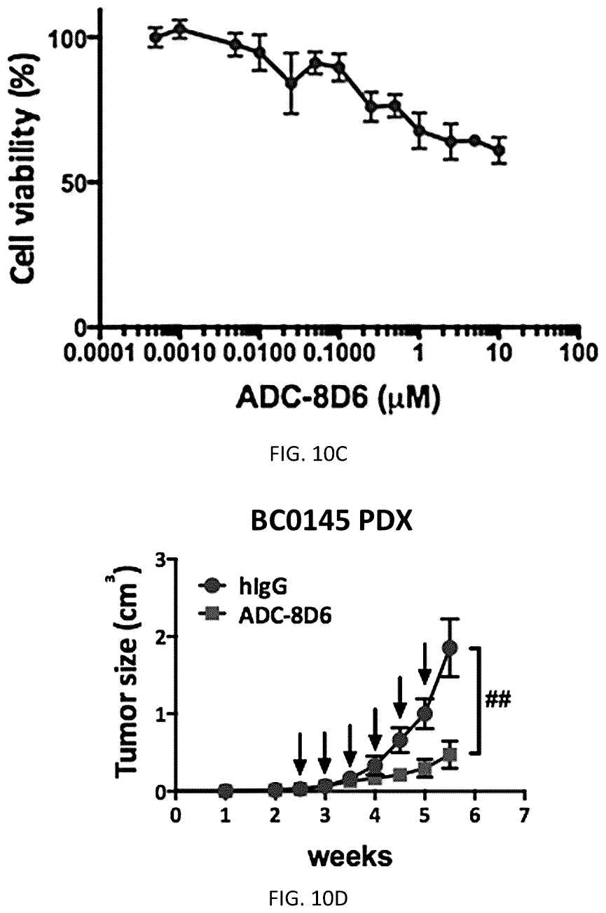

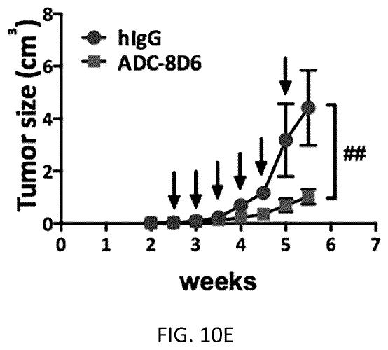

In vitro, mAb 8D6 was found to be internalized into TMCC3⁺ cells after binding with surface TMCC3 (FIG. 10A). This finding suggests that mAb 8D6 may be useful in the design of antibody-drug conjugate (ADC) to target TMCC3⁺ cancer cells. Hence, chimeric mAb8D6 was generated and conjugated with an anti-tubulin agent, MMAF, at drug-to-antibody ratio (DAR) of 3.8 (FIG. 10B). ADC-8D6 conjugated MMAF caused 60% cell death of BC0145 PDX tumor cells at 10 μM (IC₅₀=102.6 nM) in vitro (FIG. 10C). We have evaluated the anti-tumor efficacy of this first generation ADC-8D6 in two PDXs of breast cancer. Administration of ADC-8D6 conjugated with MMAF (4 mg/kg) into BC0145 or BC0634 PDX tumor-bearing mice twice per week was started when tumors reached ^{~}50 mm³. As shown in FIGS. 10D and 10E, ADC-8D6 significantly suppressed tumor growth as compared to human IgG (hIgG) treated mice (P<0.0001). In addition, treatment with ADC-8D6 diminished metastasis to lymph node and vital organs as compared with hIgG control group (Table 2). These findings show that ADC-8D6 conjugated with MMAF can inhibit tumor growth of BC0145 and BC0634 breast cancer PDXs and suppress metastasis in vivo. However, the DAR is heterogenous in this prototype ADC, which is an inherent challenge of the conventional chemical conjugation used for the development of the first generation ADC.

TABLE 2
Suppressed tumor metastasis of ADC-MMAF (8D6) in vivo
BC0145 PDX

| (Metastasis) | hIgG | 8D6 | ADC-8D6 |
|---|---|---|---|
| LN | 6/10 (60%) | 3/10 (30%) | 3/10 (30%) |
| Liver | 1/10 (10%) | 1/10 (10%) | 0/10 (0%) |
| Lung | 1/10 (10%) | 1/10 (10%) | 0/10 (0%) |

BC0634 PDX

| (metastasis) | hIgG | ADC-8D6 |
|---|---|---|
| ipsilateral-LN | 6/7 (86%) | 1/9 (11%) |
| contralateral-LN | 6/7 (86%) | 0/9 (0%) |

### Example 9 Preparation of Tri-Mannosyl-Anti-TMCC3 8D6 mAb

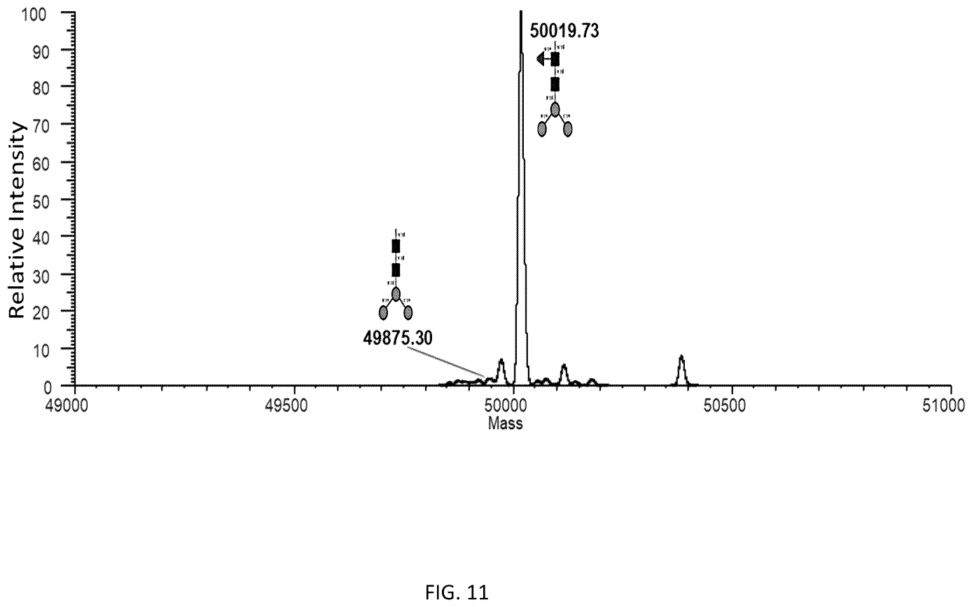

To generate ADC with homogeneous DAR with specific sites of drug-conjugation, we performed a highly efficient glyco-engineering technology platform to conjugate a tri-mannosyl core antibody for novel ADC. This platform converts the tri-mannosyl antibody to a tri-mannosyl-4GlcNAz antibody and then links the payloads to the terminal GlcNAz group at antibody's specific sites by strain-promoted azide-alkyne click chemistry (SPAAC) reaction. Anti-TMCC3 8D6 mAb was treated with β1,4-Galactosidase and α2-3,6,8 neuraminidase in 1× GlycoBuffer at 37° C. for 24 hours to remove galactose and sialic acid moieties of the N-glycan from anti-TMCC3 8D6 mAb. β1,4-Galactosidase was further added to the reactant and the reaction was allowed to perform at 37° C. for further 24 hours to obtain a G0F/G0 antibody sample. The antibody sample was purified and subjected to reduced mass chromatography analysis. As shown in FIG. 11, the major amount of the antibodies in the sample is G0F (having a heavy chain with a molecular weight of 50,019 Da) and only a small amount is G0 (without a fucose sugar; having a heavy chain with a molecular weight of 49,875 Da).

### Example 10 Preparation of Anti-TMCC3 8D6 mAb-4Az

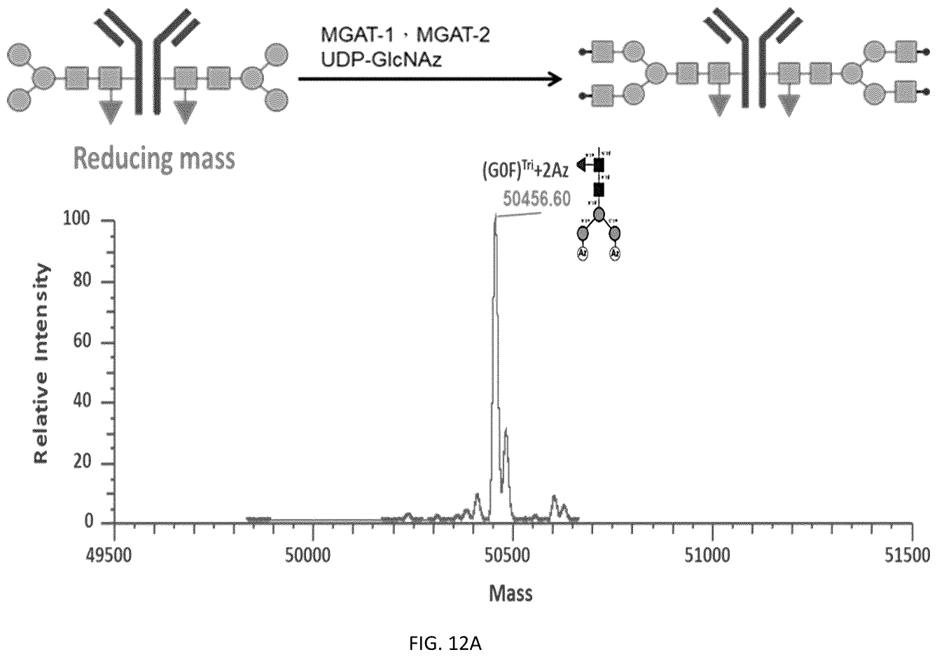

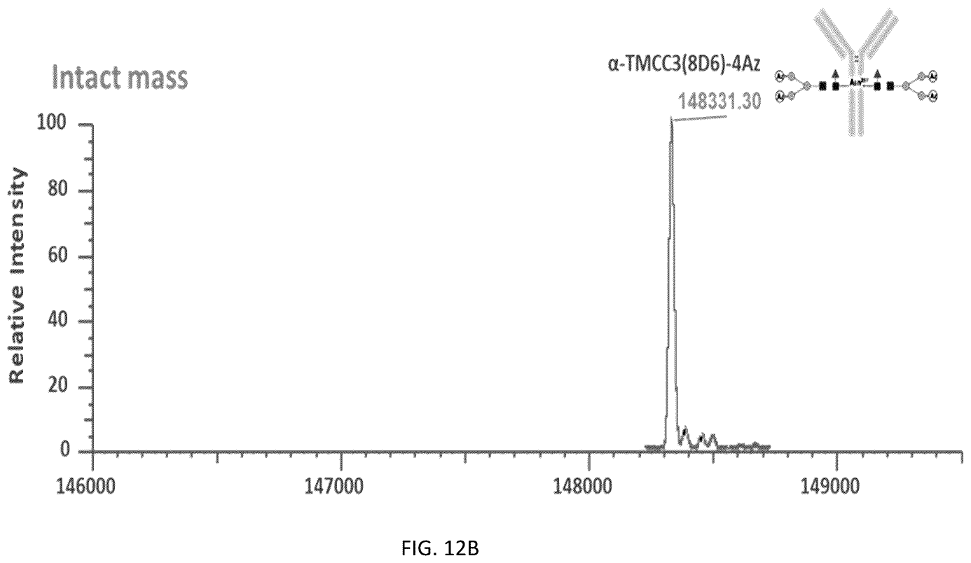

Tri-mannosyl anti-TMCC3 8D6 mAb and UDP-GlcNAz in 1× buffer SP were incubated in the presence of rabbit MGAT-1 and rat MGAT-2 at 37° C. for 16 hours. After the incubation, anti-TMCC3 8D6 mAb-4Az was subjected to reduced mass chromatography analysis and intact mass chromatography analysis. As shown in the FIG. 12A, a tri-mannosyl-anti-TMCC3 8D6 mAb-4GlcNAz antibody product was obtained, wherein the heavy chains contain two GlcNAz molecules (molecular weight of 219 Da×2=438) and each heavy chain has a molecular weight of 50,457 Da. This result suggests that through MGAT-1 and MGAT-2, GlcNAz is conjugated to α-3 mannose and α-6 mannose of each heavy chain of tri-mannosyl core anti-TMCC3 8D6 mAb. This result is further confirmed by the intact mass chromatography. As shown in FIG. 12B, compared to the whole tri-mannosyl core anti-TMCC3 8D6 mAb with the molecular weight of 147,355 Da, a G0F tri-mannose anti-TMCC3 8D6 mAb-4GlcNAz antibody product was obtained, which contains four GlcNAz molecules (molecular weight of 244 Da×4=976 Da) and has a molecular weight 148,331 Da.

### Example 11 Preparation of Anti-TMCC3 8D6 mAb-4(DBCO-vc-MMAE)

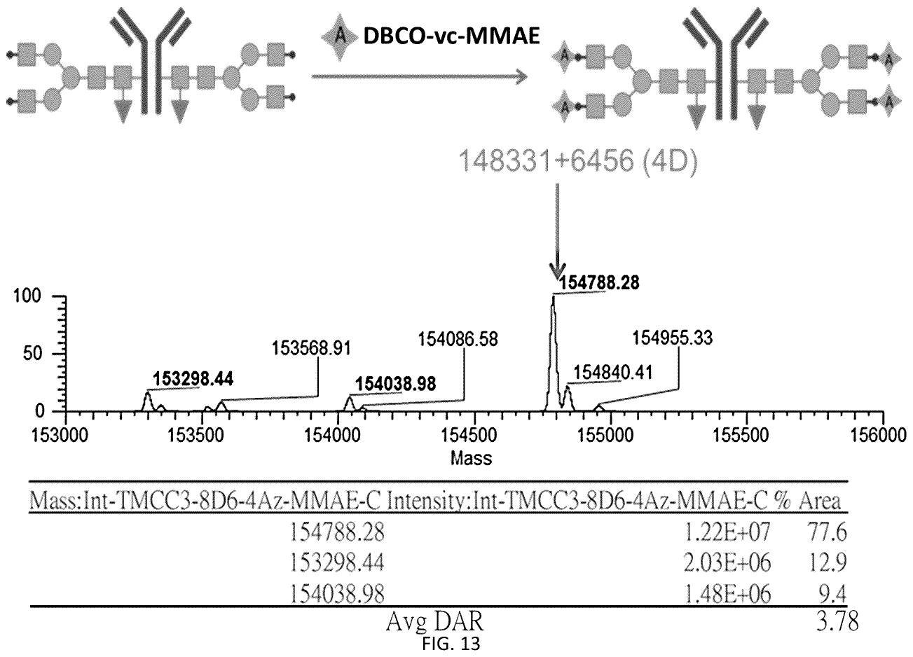

DBCO-vc-MMAE (10 mM in DMSO) was slowly added to a solution of tri-mannosyl anti-TMCC3 8D6 mAb-4GlcNAz antibody product) in MES buffer (pH 6.5) at 37° C. for 18 hours. The antibody preparation was desalted and concentrated by using the Amicon Ultra-15 centrifugal filter device with 30 kDa NMWL in Na-Citrate pH 6.5 buffer to give anti-TMCC3 8D6 mAb-4(DBCO-vc-MMAE). As shown in FIG. 13, a tri-mannosyl anti-TMCC3 8D6 mAb-4(GlcNAc-triazole-DBCO-(PEG)4-MMAE) ADC product contains four DBCO-(PEG)4-MMAE molecules (molecular weight of 1,614 Da×4=6,456 Da) (Formula I) was obtained. The molecular weight of ADC was 154,788 Da and drug-to-antibody ratio (DAR) was 3.78.

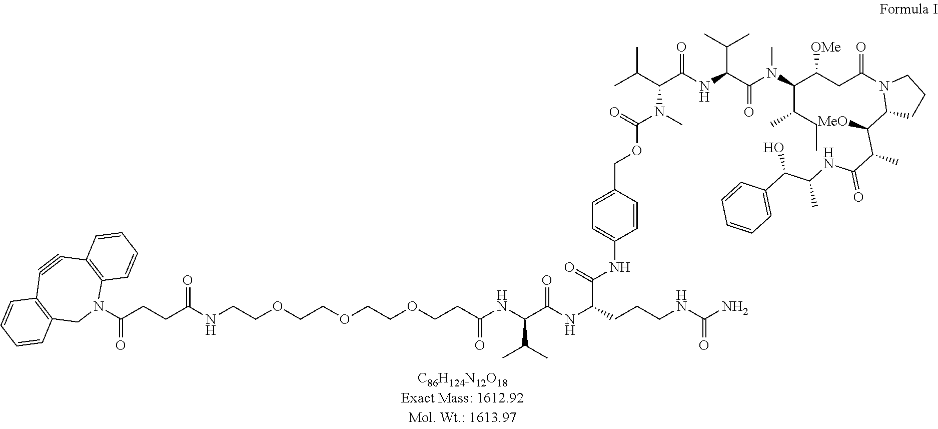 [SMILES: CC[C@H](C)[C@H]([C@@H](CC(=O)N1CCC[C@@H]1[C@H](OC)[C@H](C)C(=O)N[C@H](C)[C@@H](O)c1ccccc1)OC)N(C)C(=O)[C@@H](NC(=O)[C@@H](C(C)C)N(C)C(=O)OCc1ccc(NC(=O)[C@H](CCCNC(N)=O)NC(=O)[C@H](NC(=O)CCOCCOCCOCCNC(=O)CCC(=O)N2Cc3ccccc3C#Cc3ccccc32)C(C)C)cc1)C(C)C]

### Example 12 Anti-Cancer Efficacy of Tri-Mannosyl-MMAE 8D6-ADC

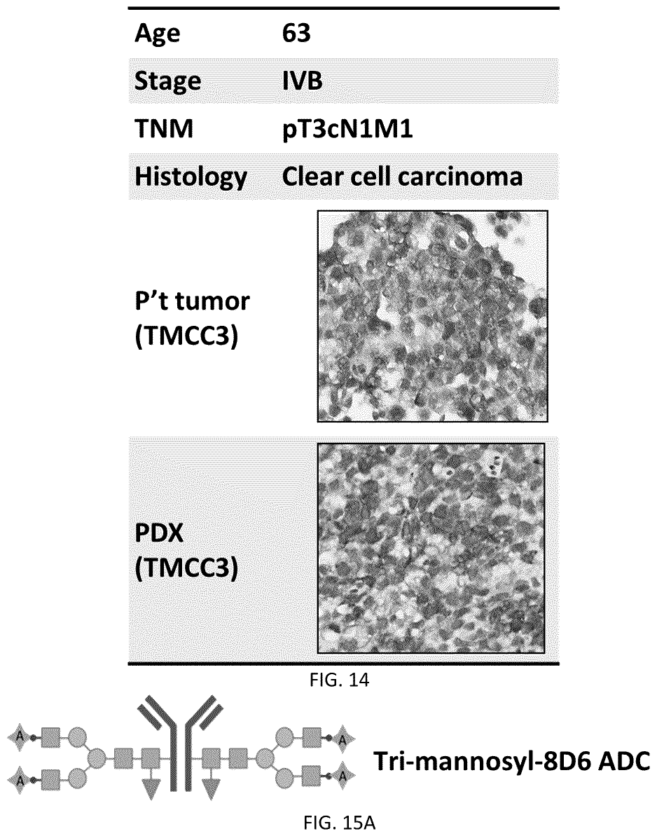

To evaluate the efficacy of tri-mannosyl ADC-8D6 on ovarian cancer, we have conducted four sets of animal studies using OC085 PDX tumor of ovarian cancer. The clinical information and drug treatment history of OC085 patient is summarized in FIG. 14. Briefly, after the diagnosis of stage 4 ovarian cancer, she received debulking surgery, followed by first-line chemotherapy with carboplatin+paclitaxel. The patient had a very short-lived disease stabilization (2.5M) and moved on to second-line chemotherapy (carboplatin+lipodox), followed by addition of Nivolumab and concomitant DC-CIK cell therapy, all of which failed to halt the disease progression. We found TMCC3 was highly expressed in patient primary tumor and PDX tumor, as shown in the FIG. 14. Thus, PDX OC085 derived from this patient who had advanced ovarian cancer with poor responses to first/second line therapy is an ideal model for our target patient population.

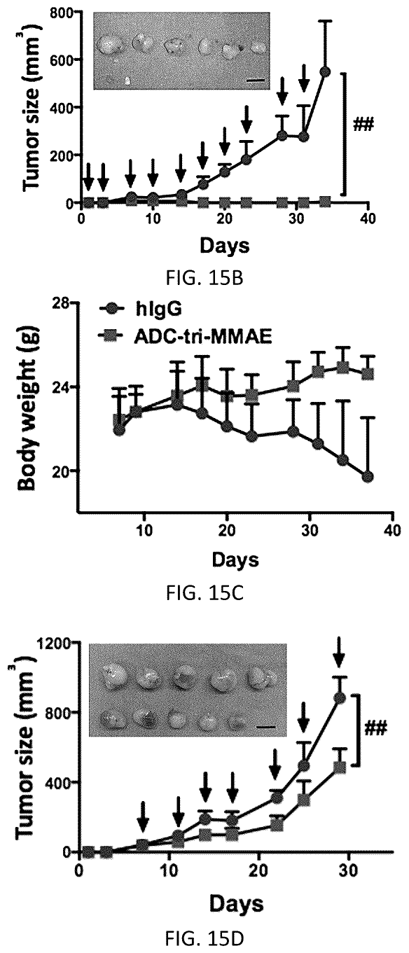

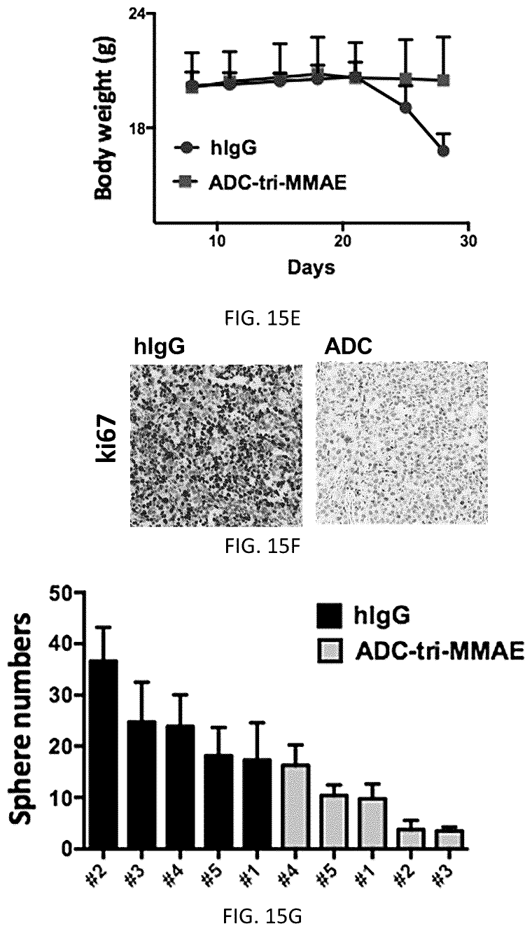

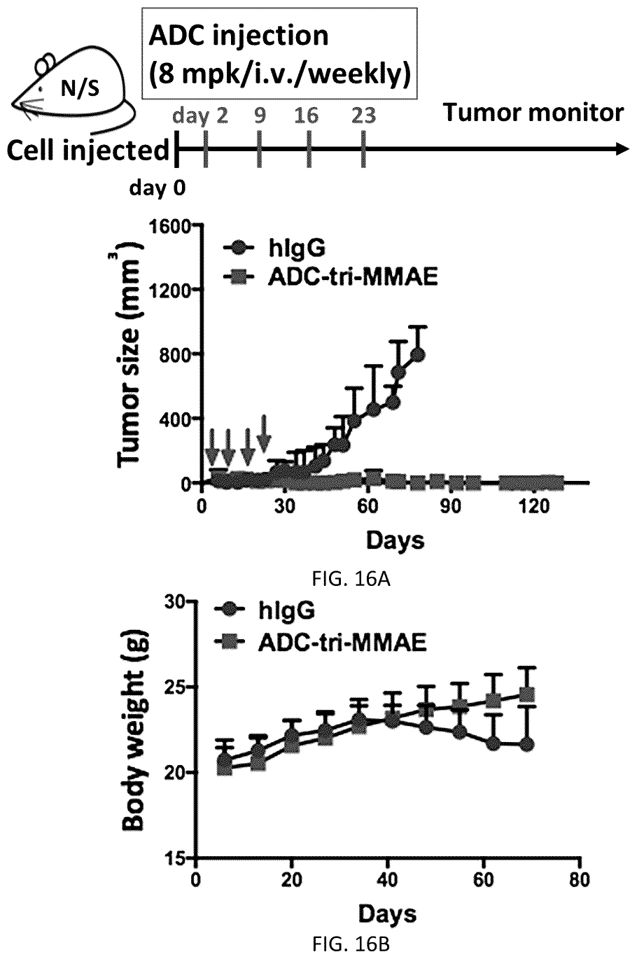

In one study, 4 mg/kg tri-mannosyl ADC-8D6-conjugated MMAE was administered twice per week, starting the same day after tumor inoculation. The ADC treatment starting the same day after tumor inoculation simulates the status of minimal residual disease (MRD), e.g. complete response after chemotherapy and/or surgical resection in patients with high-risk ovarian cancers. As shown in FIG. 15B, tumor growth was completely abrogated, in contrast to the progressive tumor growth in control mice treated with human IgG. In the second study, 4 mg/kg tri-mannosyl ADC-8D6-conjugated MMAE was not started until tumors reached ^{~}100 mm³. The tumor growth of ADC treated mice was slower than the control mice treated with human IgG (FIG. 15D) (p<0.001). As shown in FIGS. 15C and 15E, the body weights of tumor-bearing mice were not affected by ADC treatment at 4 mg/kg/dose×2/week. In addition, we analyzed ki67 expression and CSC frequencies in ADC-treated tumors upon sacrifice. As shown in FIG. 15F, lower expression of ki67 was noted in ADC-treated tumor than hIgG-treated tumors. Sphere forming capacities were lower in ADC-treated tumors as compared with hIgG control group (FIG. 15G). In the third experiment, we increased the dose of tri-mannosyl ADC-8D6-conjugated MMAE to 8 mg/kg/dose weekly (i.v. injection) for 4 weeks in OC085 ovarian PDX tumor-bearing mice starting the next day of tumor inoculation. As shown in FIG. 16A, the tumor growth of ADC-treated mice was completely abrogated, in contrast to the control mice treated with hIgG. Treatment with tri-mannosyl ADC-8D6 induced sustained tumor suppression in OC085 PDX-bearing mice up to the time of sacrifice at day 128 (FIG. 16A). Weight loss was noted in human IgG treated group, but not in ADC-treated mice (FIG. 16B).

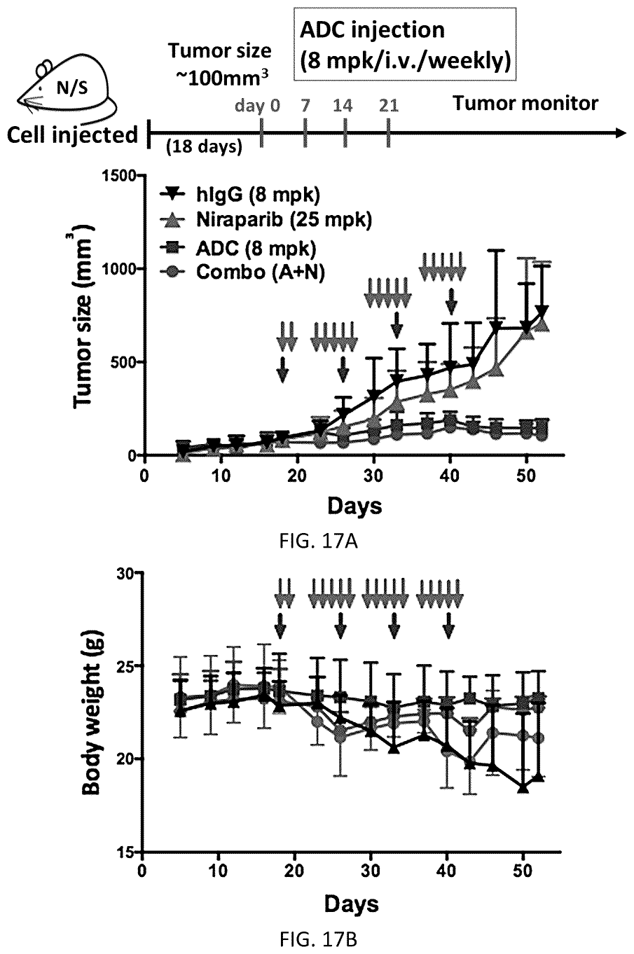

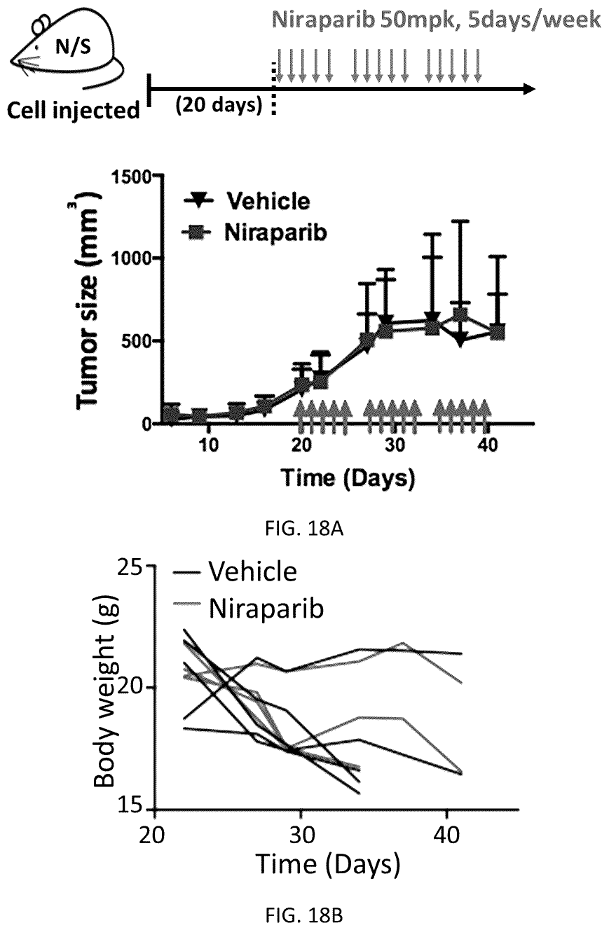

To explore the anti-cancer ability of tri-mannosyl ADC-8D6 as a single agent or in combination with niraparib (PARP inhibitor), OC085 PDX tumor-bearing mice were treated with 8 mg/kg/dose weekly tri-mannosyl ADC-8D6 (i.v. injection) alone, 25 mg/kg/day niraparib (oral gavage, 5 days/week) alone or in combination when tumors reach ^{~}100 mm³. As shown in FIG. 17A, tumor growth of tri-mannosyl ADC-8D6 treated mice was negligible, in contrast to the robust tumor growth in human IgG treated mice (p<0.001). The dosage of 25 mpk niraparib was chosen based on the report that this is a therapeutic dose in mice bearing ovarian tumor and cholangiocarcinoma (Bezrookove, V., et al. Cancers 13, 4405 (2021); Meng, J., et al. Journal of Translational Medicine 19 (2021)). However, in OC085 PDX bearing mice treatment with 25 mpk niraparib only reduced tumor growth slightly but did not reach statistical significance. Combination of ADC-8D6 and niraparib abrogated the tumor growth similar to the mice treated with ADC-8D6 alone (FIG. 17A). In this experiment, weight loss was not observed in mice treated with ADC-8D6 alone or in combination with niraparib (FIG. 17B). Higher dose of niraparib was tested further. As shown in FIG. 18, administration of 50 mg/kg/day niraparib did not suppress tumor growth but caused debilitated condition with significant body weight loss in mice bearing OC085 PDX tumors. These results showed tri-mannosyl ADC-8D6 has greater anti-cancer activity and fewer side effects than niraparib in vivo.

TMCC3 has been shown to be crucial for CSCs of breast and ovarian cancers, and mAb8D6 has been shown to be an anti-TMCC3 specific monoclonal antibody with theranostic potentials. In our six animal experiments described above, we have demonstrated that ADC-8D6 exhibited not only anti-cancer activities in breast cancer PDX (BC0145 & BC0634) and ovarian PDX (OC085) tumors, but also CSC-targeting capacity in vivo. In the comparison experiment of ADC-8D6 vs. therapeutic dose of niraparib, ADC-8D6 clearly shows better anti-tumor efficacy than niraparib in ovarian PDX (OC085) tumors. These results provide the impetus to further develop anti-TMCC3/ADC into an anti-cancer agent targeting ovarian cancer stem cells.

While the present disclosure has been described in conjunction with the specific embodiments set forth, many alternatives thereto and modifications and variations thereof will be apparent to those of ordinary skill in the art. All such alternatives, modifications and variations are regarded as falling within the scope of the present disclosure.

What is claimed is:

1. An antibody or antigen-binding fragment thereof that is specific for an epitope in transmembrane and coiled-coil domain family 3 (TMCC3); wherein the antibody or antigen-binding fragment thereof comprises complementarity determining regions (CDRs) of a heavy chain variable region and CDRs of a light chain variable region,
wherein the CDRs of the heavy chain variable region comprise:
CDRH1 of an amino acid sequence of SEQ ID NO: 3, CDRH2 of an amino acid sequence of SEQ ID NO: 4, and CDRH3 of an amino acid sequence of SEQ ID NO: 5; and
wherein the CDRs of the light chain variable region comprise:
CDRL1 of an amino acid sequence of SEQ ID NO: 6, CDRL2 of an amino acid sequence of SEQ ID NO: 7, and CDRL3 of an amino acid sequence of SEQ ID NO: 8.

2. The antibody or antigen-binding fragment thereof according to claim 1, wherein the heavy chain variable region comprises the amino acid sequence of SEQ ID NO: 1 or a substantially similar sequence having at least 95% sequence identity; and/or the light chain variable region comprises the amino acid sequence of SEQ ID NO: 2 or a substantially similar sequence having at least 95% sequence identity.

3. The antibody or antigen-binding fragment thereof according to claim 1, wherein the antibody is an Fab fragment, an F(ab′) 2 fragment, an ScFv fragment, a monoclonal antibody, a chimeric antibody, a nanobody, a humanized antibody or a human antibody.

4. The antibody or antigen-binding fragment thereof according to claim 1, wherein the antibody is multispecific.

5. The antibody or antigen-binding fragment thereof according to claim 4, which is linked to a second antibody or antigen-binding fragment thereof that is specific for a second epitope.

6. The antibody or antigen-binding fragment thereof according to claim 1, which is conjugated with a therapeutic agent, wherein the therapeutic agent is selected from antimetabolites, alkylating agents, alkylating-like agents, DNA minor groove alkylating agents, antibiotics, antimitotic agents, topoisomerase inhibitors, proteasome inhibitors, radioisotopes, DM1, DM3, DM4, monomethyl auristatin E (MMAE), and monomethyl auristatin F (MMAF).

7. (canceled)

8. (canceled)

9. The antibody or antigen-binding fragment thereof according to claim 1, which is expressed on a surface of a cell.

10. The antibody or antigen-binding fragment thereof according to claim 9, wherein the cell is an immune cell, a cancer stem cell or a stem cell.

11. (canceled)

12. A vector encoding the antibody or antigen-binding fragment thereof according to claim 1.

13. A genetically engineered cell expressing the antibody or antigen-binding fragment thereof according to claim 1.

14. (canceled)

15. A pharmaceutical composition comprising the antibody or antigen-binding fragment thereof according to claim 1 and a pharmaceutically acceptable carrier.

16. A vessel or injection device comprising the antibody or antigen-binding fragment thereof according to claim 1.

17. A pharmaceutical composition for use in treating, prophylactic treating and/or preventing a disease related to a TMCC3-mediated signal in a subject in need thereof, comprising an effective amount of the antibody or antigen-binding fragment thereof according to claim 1.

18. The pharmaceutical composition according to claim 17, wherein the disease is a cancer.

19. The pharmaceutical composition according to claim 18, wherein the cancer is a solid cancer.

20. The pharmaceutical composition according to claim 17, wherein the cancer is lung cancer, breast cancer, ovary cancer, pancreas cancer, bile duct cancer, gallbladder cancer, prostate cancer, or colorectal cancer.

21. (canceled)

22. (canceled)

23. A method for detecting TMCC3, a cancer stem cell, or a cancer in a sample, comprising: contacting the sample with the antibody or antigen-binding fragment thereof according to claim 1, and assessing a level of expression of TMCC3 in the sample with the antibody or antigen-binding fragment thereof, wherein an increase in the level of expression of TMCC3 as compared to a standard indicates the presence of cancer stem cells in the sample.

24. (canceled)

25. The method according to claim 23, wherein the cancer stem cell is a hematopoietic, epidermal, breast, ovary, lung, pancreas, prostate, brain, colon, bone marrow, or lymph cancer stem cell.

26. A kit for detecting TMCC3 or a cancer in a sample, wherein the kit comprises the antibody or antigen-binding fragment thereof according to claim 1.

27. A method for detecting a predisposition to a cancer or predicting likelihood, treatment response, prognosis or recurrence of the cancer in a subject, comprising
assessing a level of expression of TMCC3 in a sample with the antibody or antigen-binding fragment thereof according to claim 1, wherein an increase in the level of expression of TMCC3 as compared to a standard indicates the presence of cancer stem cells in the sample.
